(19) United States
(12) Patent Application Publication
(10) Pub. No.: US 2003/0077817 A1
(43) Pub. Date: Apr. 24, 2003

(54) Microfermentor device and cell based screening method

(76) Inventors: Andrey J. Zarur, Winchester, MA (US); H. Brett Schreyer, Tewksbury, MA (US); Lawrence Fama, LaHonda, CA (US); Anne Heibel, San Francisco, CA (US)

Correspondence Address: FISH & RICHARDSON PC, 225 FRANKLIN ST, BOSTON, MA, 02110, US

(21) Appl. No.: 10/119,917

(22) Filed: Apr. 10, 2002

Related U.S. Application Data
(60) Provisional application No. 60/282,741, filed on Apr. 10, 2001.

(51) Int. Cl. C12M 3/00

(52) U.S. Cl. 435/305.2; 435/288.5; 435/288.7

(57) ABSTRACT

A microfermentor device that can be used for a wide variety of purposes is described. The microfermentor device includes one or more cell growth chambers having a volume of less than 1 ml. The microfermentor device can be used to grow cells used for the production of useful compounds, e.g., therapeutic proteins, antibodies or small molecule drugs. The microfermentor device can also be used in various high-throughput screening assays. For example, the microfermentor device can be used to screen compounds to assess their effect on cell growth and/or a normal or abnormal biological function of a cell and/or their effect on the expression of a protein expressed by the cell. The device can also be used to investigate the effect of various environmental factors on cell growth, biological function or production of a cell product. The device, including various controlling components and sensing components can be microfabricated on a support material.

### RELATED APPLICATION INFORMATION

This application claims priority from provisional application serial No. 60/282,741, filed Apr. 10, 2001.

### TECHNICAL FIELD

This invention relates to a microfermention device, and more particularly to a microfermentation device that is microfabricated on a solid substrate. The invention also relates to screening and testing methods employing such microfermentation devices.

### BACKGROUND

Cells grown in culture produce many valuable drugs and other compounds. Very often it is important to identify the specific cell line, growth conditions and chemical or biological agents that permit optimized production of the desirable material by the cultured cells. Optimization of these various factors is important for the cost-effective production of needed quantities of the desired material. However, large scale screening of the various factors that might influence production is costly and time-consuming because a very large number of individual cell cultures must be prepared, grown and monitored. Micro-hollow fiber bioreactors have been proposed as means for screening many different cell lines and conditions (see, e.g., U.S. Pat. No. 6,001,585). Nevertheless there is a need for sophisticated systems that are suitable for automated high throughput screening of cell culturing conditions.

The major steps in drug development (drug target identification, lead development, target analysis and screening, bioprocessing and compound screening, and regulatory approval) can take 12-17 years and cost 250-650 million (U.S.) dollars. Recent advances in high-throughput screening techniques allow for testing of the interaction of literally hundreds of thousands of leads or candidate compounds against specific biological molecules, such as enzymes and other proteins. However, these techniques are limited in that the interactions between the test compound and the biological molecule are evaluated in a model system that generally differs considerably from the real biological system in which the drug would ultimately be used. For example, systems commonly used in traditional high-throughput screening can contain a biological molecule in solution or cell cultures in batch. If the drug interacts with an intracellular enzyme or receptor, then those tests often offer limited or irrelevant information about the real-life effects. As a result, high-throughput screening tests often have to be validated in cell cultures or animal models. Both systems are labor intensive and difficult or impossible to automate. In addition to these difficulties, the use of animals in drug screening and testing is becoming less socially acceptable in the United States, Europe and elsewhere. Thus there is a need in the drug discovery process for a rapid, high throughput, and cost effective screening process that simulates as closely as possible the biological environment in which the drug is expected to act.

### SUMMARY

The invention features a microfermentor device that can be used for a wide variety of purposes. For example, the microfermentor device can be used to grow cells used for the production of useful compounds, e.g., therapeutic proteins, antibodies or small molecule drugs. The microfermentor device can also be used in various high-throughput screening assays. For example, the microfermentor device can be used to screen compounds to assess their effect on cell growth and/or a normal or abnormal biological function of a cell and/or their effect on the expression of a protein expressed by the cell. The device can also be used to investigate the effect of various environmental factors on cell growth, biological function, or production of a cell product.

The microfermentor device is produced by microfabrication and can contain one or many cell culture chambers. The device includes controllers, sensors, microfluidic channels, and microelectronic devices to monitor and control the environment within the cell culture chambers. The various controllers, sensors, microfluidic channels, and microelectronic devices can serve one or more cell culture chambers. The devices allows for monitoring of real-time responses of cells to a biologically active compound or a combination of compounds and to environmental factors. Because the device can include numerous cell culture chambers and because several devices can be operate in parallel, the microfermentor device of the invention allows for high throughput screening of large numbers of compounds, cells, and growth conditions.

In essence, the microfermentor device of the invention has many or all of the capabilities of an industrial fermentor. It provides a well-mixed culture environment with controllable temperature, pH, dissolved oxygen concentration, and nutrient levels, but does so on a micro scale that permits cost-effective, highly automated, highly controllable, and highly monitored screening and testing.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a cross sectional view of a cell growth chamber of a microfermentor of the invention showing a portion of each of three associated microchannels.

FIG. 2 is a cross sectional view of a gas headspace portion of a cell growth chamber of the invention showing a portion of each of two associated microchannels.

### DETAILED DESCRIPTION

The microfermentor devices of the invention are designed to facilitate very small scale culturing of cells or tissues. A single microfermentor device can contain a number of separate cell culture chambers. Each cell culture chamber can be individually controlled and monitored. Thus, a single microfermentor device or an array of microfermentor devices can be used to simultaneously grow a variety of cells under a variety of conditions. Thus, the microfermentor device of the invention is useful for high throughput screening of many cell types and growth conditions.

The microfermentor device of the invention can be integrated into a microreactor system such as that described in PCT Publication WO 01/68257 A1, hereby incorporated by reference.

The microfermentor device of the invention can be constructed using standard microfabrication processes (e.g., chemical wet etching, chemical vapor deposition, deep reactive ion etching, anodic bonding, and LIGA) and is built on a suitable substrate (e.g., glass, quartz, silicon wafers, polymer, and metal) for microfabrication. The substrate material can be rigid, semi-rigid or flexible. It can be opaque, semi-opaque or transparent. In some cases the substrate is layered and uses combinations of different types of materials. Thus, a base layer might be opaque and a top layer might be transparent or include transparent or semi-transparent portion.

The microfermentor device can be provided with microvalves and micropumps that are fabricated on the solid support or chip using standard microfabrication techniques similar to those used to create semiconductors (see Madou, Fundamentals of Microfabrication, CRC Press, Boca Raton, Fla., 1997; Maluf An Introduction of Micromechanical Systems Engineering, Artech House, Boston, Mass. 2000).

The microfermentor device of the invention can include one or many (e.g., 5, 10, 20, 50, 100, 500, 1000 or more) separate cell culture chambers in a single unit. An array of many microfermentor devices (e.g., 100, 200, 500, 1000 or more) can be operated in parallel. The microfermentor devices are monitored and controlled automatically using robotics. The consistency and scalability of the microfermentor system allows to screen many compounds or to test many different growth conditions or cell lines simultaneously. The microfermentors can provide flow, oxygen and nutrient distribution properties similar to those found in living tissue. Thus it can be used for high-throughput, automated screening under conditions that are closer to in vivo than those provided by batch culture-like, well-plate systems.

The microfermentor device is preferably fabricated on a solid support. Thus, the cells growth chamber, along with the various elements that allow material to be added to or withdrawn from the chamber and all of the elements desired for control and monitoring of the chamber are fabricated on or integrated into the solid support.

Each microfermentor includes a chamber where the cells are cultured. The reaction chamber is provided with at least one fluid inlet port and at least one fluid outlet port. The volume of the reaction chamber is less than about 2 ml or smaller (e.g., less than about 1 ml, 500 μl, 300 μl, 200 μl, 100 μl, 50 μl, 10 μl, 5 μl, or 1 μl). The chamber can be partially or completely lined with a support material to which cells can adhere. Similarly, the chamber can be partially or completely filled with a support matrix to which cells can adhere.

Because growing cells must be provided with a source of oxygen, and other gasses (e.g., nitrogen and carbon dioxide), there is a gas headspace associated with reaction chamber. The gas headspace can be located above the chamber, separated by a gas permeable membrane. In most case the membrane will be relatively impermeable to water vapor. The gas headspace is provided with a gas inlet port and a gas outlet port. The ports are connected to microchannels that can be provided with microfabricated pumps and valves. The channels can also be provided with microfabricated flow meters. The gas headspace and the microchannels can also include various sensors for monitoring temperature and other conditions.

The microfermentor device is provided with various sensors. For example, each chamber can be provided with sensors for measuring optical density, pH, dissolved oxygen concentration, temperature, and glucose. Sensors can be used to monitor the level of a desired product synthesized by the cells, e.g., a desired protein product. The sensors can be external to or integrated into the substrate of the microfermentor device. It can be desirable to use sensors that do not need to come into physical contact with the cell culture itself. Thus, it can be desirable to use remote sensing techniques, e.g., techniques based on optical detection of an indicator compound. For example, Ocean Optics Inc. (Dunedin, Fla.) provides fiber optic probes and spectrometers for the measurement of pH and dissolved oxygen concentration. These devices rely on the detection of chromogenic substances. For pH measurement, buffered chromogenic substrates are available. The color and intensity of the chromogenic substrate, which reflects the pH of the medium, is measured using a fiber optic probe and spectrometer. Dissolved oxygen concentration can be measured using a similar color based procedure. In addition to remote measurement methods, more direct sensors can be used, e.g., micro-pH, micro-dissolved oxygen probes, and micro-thermocouples for measurement of temperature.

The devise can include sensors that monitor the gas phase of the cell culture chamber. Other sensors can monitor the various microfluidic channels connected (directly or indirectly) to the cell culture chamber. The sensors can measure temperature, flow, and other parameters.

In addition to the various sensing elements described above, the device includes a number of control elements. Thus, the temperature of the cell culture chamber can be controlled using heat exchanges that are in contact with the substrate in which the chamber resides. The pH of the cell culture can be controlled by the addition of chemicals. The level of dissolved oxygen can be controlled by adjusting the flow of oxygen into the cell culture chamber.

The cell culture chamber is provided with at least one port for the aseptic introduction of various compounds (e.g., nutrients, test compound, candidate therapeutic agents, growth factors, and biological modifiers such as growth factors).

Computerized control and expert systems can be used to monitor and control the operation of the microfermentor device. This permits the monitoring and control of multiple cell growth chambers and multiple microfermentor devices. Each cell culture chamber can be monitored and controlled individually. Alternatively, cell culture chambers can be monitored and controlled in groups. For example, ten chambers in a device can be held at one temperature and ten other chambers in the device can be held at a different temperature. It is also possible to have more complex control and monitoring arrangements. For example, where there are a plurality of cell culture chambers, subset A can be held at one temperature and subset B can be held at a different temperature. At the same time subset α, which contains members of subset A and subset B can have a first test compound added to them, while members of subset β, which also contains members of subset A and subset B can have a second test compound added to them. In this manner it is possible to provide a very large number of cell culture chambers in which cells are grown under differing conditions. It is also possible to alter the pattern of control and monitoring over time. Thus, two chambers that are monitored and control identically at a first time point can be separately monitored and controlled at a second time point. The control and monitoring can be preset and automated and can include provisions for manual over-ride.

Various types of cells can be grown in the microfermentor device. For example, bacteria, fungi, plant cells, insect cells, or any line of mammalian cells. The entire device or at least all of the portions coming into contact with the cells being cultured can be sterilized either chemically, by heating, by irradiation, or by other suitable means. The cells can be immobilized on a support that coats all or a portion of the interior of the cell culture chamber or on a filling material that partially or completely fills the cell growth chamber.

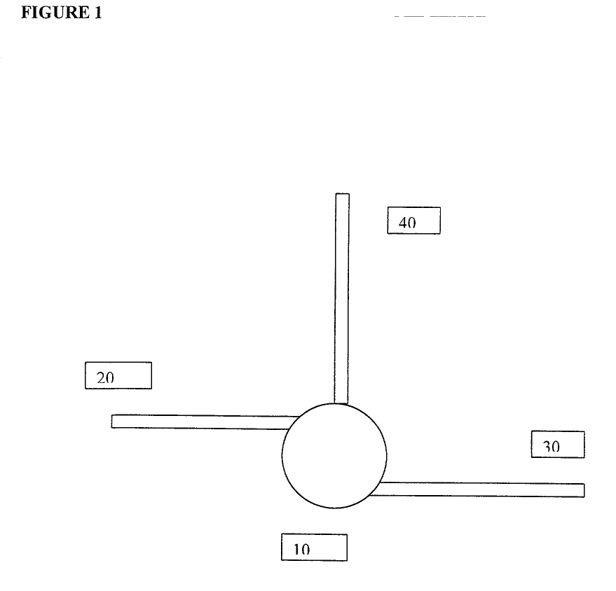

FIG. 1 depicts a cross-sectional view of the cell culture chamber of a microfermentor device of the invention. The cell culture chamber 10 is a cylinder 7000 μm in diameter and 100 μm in height having a total volume of 3.85 μL. The chamber is fluidly connected to three microchannels. The first microchannel 20 is 400 μm wide by 100 μm deep and serves as a liquid inlet. The second microchannel 30 has similar dimension and serves as a liquid outlet. The third micro-channel 40 is 200 μm wide by 100 μm deep. This microchannel can be used to introduce cells or any desired material into the chamber. The three microchannels and the cell culture chamber are etched into a solid support material.

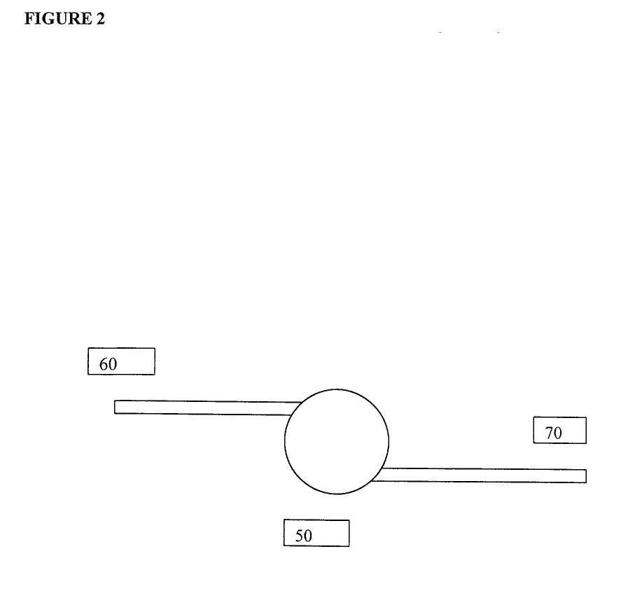

FIG. 2 depicts a cross-sectional view of a gas headspace portion associated with a cell culture chamber. This allows a continuous supply of air to pass through the microfermentor. A cylindrical chamber 50 that is 7 mm in diameter and 50 μm in height is etched in glass along with a gas inlet microchanel 60 and gas outlet microchannel 70, both of which are 50 μm wide by 50 μm deep. The cylindrical chamber of the gas headspace portion is matched over the cell culture chamber. The two halves can then be bonded together so as to form a tight seal.

To prevent the air flowing through the gas headspace from removing liquid in the cell culture chamber, a membrane is placed in so as to separate the gas headspace from the liquid filled bioreactor. The membrane retards passage of water and allows for the passage of air.

The various microchannels are connected to supply units or waste units. These units as well a mixing devices, control valves, pumps, sensors, and monitoring devices can be integrated into the substrate in which the cell culture chamber is built or can be externally provided. The entire assembly can be placed above or below a heat exchanged (or sandwiched between two heat exchangers) to control the temperature of the unit.

The microfermentor device of the invention can be used to produce a valuable product, e.g., a therapeutic protein, an enzyme, a vitamin, an antibiotic, or a small molecule drug. By operating microfermentors in parallel significant quantities of a desired product can be prepared. The microfermentor can be used to screen compounds or growth conditions for their effect on the production of a desired product or on the growth of a cell. In addition, many different cell types or clones can be screened at one time.

### EXAMPLE 1

The microfermentor device of the invention can be used to examine the effect of chemical agent A on fermentation of a bacterium. Twelve microfermentors, each bearing a single cell growth chamber are aligned in parallel. The microfermentors are sterilized and sterile growth media is pumped into each microfermentor through a fluid delivery system. Six microfermentors receive a measured aliquot of chemical agent A through the fluid delivery system and the remaining six do not. Having six microfermentors for each case provides a measure of redundancy for statistical purposes. Each of the 12 microfermentors is inoculated with a volume of concentrated cells, the volume being about 1/20 to 1/10 the volume of the microfermentor, and the cells being a pure culture of the bacteria of choice, e.g., Escherichia coli. A supply of sterile air is continuously added to the microfermentor through the fluid delivery system to provide a source of oxygen for the microorganisms. The growth of the microorganisms is monitored in each of the 12 microfermentors by measuring pH, dissolved oxygen concentration, and cell concentration with respect to time through the use of appropriate sensors in the microfermentors. Just as with a bench scale fermentor, the microfermentor can control various aspects of the cell culture environment. For example, through the use of heat exchangers, addition of chemicals, and airflow rate, the microfermentor can control temperature, pH, and dissolved oxygen concentration, respectively. At the end of the fermentation (when cells reach stationary phase, i.e. are no longer dividing), average cell growth rate and average final cell concentration can be computed for the six microfermentors with chemical agent A and for the six microfermentors without. By comparing these averages, chemical agent A can be said to enhance cell growth, have no significant effect, or hinder cell growth.

### EXAMPLE 2

The microfermentor device of the invention can provide an environment to grow cells or tissue that closely resembles of that found in humans or mammals. With respect to drug screening, the microfermentor can monitor responses of cells to a drug candidate. These responses can include increase or decrease in cell growth rate, cell metabolic changes, cell physiological changes, or changes in uptake or release of biological molecules. With many microfermentors operating in parallel, different cell lines can be tested along with screening multiple drug candidates or various drug combinations. By incorporating necessary electronics and software to monitor and control an array of microfermentors, the screening process can be automated.

Twenty microfermentors each containing a single cell culture well are sterilized. Sterile animal cell culture media is pumped into each of the microfermentors through the fluid delivery system. Each microfermentor is then inoculated with mammalian cells that are genetically engineered to produce a therapeutic protein. The cells are allowed to grow to production stage all the while their growth and environment is monitored by sensors in the microfermentor. The microfermentor, through control of temperature, pH, and air flow rate, is able to maintain an optimal environment for growth of the cells. Once at production stage, the microfermentors are separated into four groups of five. Three of the four groups receive various cocktails of inducers for the therapeutic protein while the fourth group serves as a control and thus receives no inducers. The inducer mixtures are injected through the fluid delivery system. All of the microfermentors are injected with a marker chemical that binds with the therapeutic protein. When the culture is irradiated with light at a wavelength that excites the bound marker chemical, the chemical then fluoresces, and the intensity of fluorescence is proportional to the concentration of therapeutic protein in the culture. Both the irradiated light and the fluorescent signal are passed through the detection window covering the microfermentor chamber. The fluorescent signal is picked up by a photodectector outside the microfermentor. Production of the therapeutic protein is monitored for each of the four groups, and at the end of production, average production rates and average total production can be computed for each group. Comparison of production between the four groups can then determine the effectiveness of the various inducers on protein production.

A number of embodiments of the invention have been described. Nevertheless, it will be understood that various modifications may be made without departing from the spirit and scope of the invention. Accordingly, other embodiments are within the scope of the following claims.

What is claimed is:

1. A microfermentor device comprising:
a substrate having at least one surface;
a cell culture chamber having a volume of less than about 1000 μl fabricated into the surface of the substrate;
at least a first and a second channel fabricated into the surface of the substrate and fluidly connected to the chamber; and
an optical sensor in optical communication with the chamber.

2. The device of claim 1 wherein the chamber has a volume of less than 100 μl.

3. The device of claim 1 wherein the chamber has a volume of less than 10 μl.

4. The device of claim 1 wherein the chamber has a volume of less than 1 μl.

5. The device of claim 1 wherein the first channel is fluidly connected to a mixing chamber.

6. The device of claim 5 wherein the mixing chamber is fluidly connected to a plurality of inlet channels.

7. The device of claim 6 wherein the mixing chamber and the plurality of inlet channels are fabricated in the surface of the substrate,

8. The device of claim 1 wherein the substrate is formed of a material selected from the group consisting of glass, silicon, metal, and a polymer.

9. The device of claim 1 wherein the chamber is lined with a material to which mammalian cells adhere.

10. The device of claim 1 wherein the chamber contains a matrix material to which cells adhere.

11. The device of claim 1 further comprising a sensor for monitoring the temperature within the chamber.

12. The device of claim 1 further comprising a sensor for monitoring the pH within the chamber.

13. The device of claim 1 further comprising a sensor for monitoring the pressure within the chamber.

14. The device of claim 1 further comprising a sensor for monitoring the optical density within the chamber.

15. The device of claim 1 further comprising a sensor for monitoring the glucose concentration within the chamber.

16. The device of claim 1 comprising at least 10 chambers.

17. The device of claim 16 comprising at least 20 chambers.

18. The device of claim 17 comprising at least 50 chambers.

19. The device of claim 18 comprising at least 100 chambers.

20. A method for screening a plurality of test compounds, the method comprising:
providing substrate having a surface into which is fabricated a plurality of cell culture chambers having a volume less than about 1000 μl and containing cells, each of the cell culture chambers being fluidly connected to at least a first and a second microchannel fabricated into the surface of the substrate; culture chambers being fluidly connected to at least a first and a second microchannel fabricated into the surface of the substrate;
introducing each of the plurality of test compounds into at least one of the plurality of cell culture chambers; and
monitoring the effect of each of the plurality of test compounds on a biological response of the cells.

21. The method of claim 20 wherein the biological response is cell growth.

22. The method of claim 20 wherein the biological response is production by the cells of a selected molecule.

23. The method of claim 20 wherein the biological response is uptake by the cells of a selected molecule.

24. The method of claim 20 wherein the step of monitoring comprises measuring a fluorescent signal that is influenced by the biological response.

25. The method of claim 20 wherein the device comprises at least a first and a second cell culture chamber, the first cell chamber containing a first type of cell and the second cell culture chamber containing a second type of cell.
